(19) United States
(12) Patent Application Publication
(10) Pub. No.: US 2012/0309046 A1
(43) Pub. Date: Dec. 6, 2012

(54) METHOD AND DEVICE FOR DETERMINING THE VIABILITY OF A PLANT SAMPLE

(75) Inventors: Rolando Garcia González, Talca (CL); Karla Andrea Quiroz Bravo, Talca (CL); Peter Douglas Savaria Caligari, Talca (CL)

(73) Assignee: UNIVERSIDAD DE TALCA, Talca (CL)

(21) Appl. No.: 13/576,964

(22) PCT Filed: Jan. 28, 2011
(86) PCT No.: PCT/CL2011/000010

§ 371 Date: Aug. 3, 2012

(30) Foreign Application Priority Data
Feb. 4, 2010 (CL) 099-2010

(51) Int. Cl. G01N 21/17; C12M 1/34; C12Q 1/02

(52) U.S. Cl. 435/29; 435/287.1; 435/288.3

(57) ABSTRACT

A method for determining the viability of a plant sample includes providing a viability detection device containing a solid or semisolid culture medium suitable for the nutritional requirements of a plant sample, wherein the culture medium has a starch supplement; growing the plant tissue in the viability detection device from the previous step; removing the plant tissue sample from the viability detection device; and revealing the viability detection device.

### RELATED APPLICATIONS

This is a §371 of International Application No. PCT/CL2011/000010, with an international filing date of Jan. 28, 2011 (WO 2011/094888 A1, published Aug. 11, 2011), which is based on Chilean Patent Application No. 099-2010, filed Feb. 4, 2010, the subject matter of which is incorporated by reference.

### TECHNICAL FIELD

This disclosure relates to a viability detection method for plant cells, tissues and plants which does not cause damage in the plant sample under study, allowing its normal growth after being submitted to evaluation. The method disclosed is based on the determination of the activity of the alpha-amylase enzyme present in plant tissues.

This disclosure also relates to a viability detection device and the suitable revealing system.

### BACKGROUND

The development of in vitro plant culturing techniques permitted the creation of a biotechnological industry oriented to the production and commercialization of plants at a world level. One of the critical points in the development of this industry is the determination of the viability of the tissues with which one works, a determinant factor for methodological efficiencies and profitability of the production. On the other side, at the level of research studies, physiological and biotechnological studies, cell viability represents an important parameter associated to the response to biotic or abiotic stress, generation of genetically engineered plants among other uses. Viability has been an essential parameter in the assessment of adaptation to different types of stress, such as cold tolerance (Leborgne et al. 1995) and in responses to freezing, heat or high salinity (Ishikawa et al. 1995).

The methods used for assessing viability of plant cells or tissue are mostly invasive and destructive, based on the irreversible staining of plant cells or tissues, without the possibility of recovering and reintroducing them into the productive system. On the other side, these methods are usually complex and costly for the plant producing industry. In general terms, the methods used for measuring viability may be classified in two groups: those that only stain dead cells and those that only color the living cells, in this latter case, the color is normally a product of metabolic activity (Widholm, 1972). The most used stains for dead cells are Evans blue, bromophenol blue, methylene blue and phenosafranin, whereas fluorescein diacetate (FDA) is used for living cells.

Another viability evaluation method includes determination of enzymes such as reductases and esterases. Reductase activity is determined spectrometrically measuring the absorbance of formazan, which is a reduction product of 2,3,5-triphenyltetrazolium chloride (Towill and Mazur, 1974). The determination of esterase activity uses its ability for hydrolyzing the fluorogenic substrate fluorescein diacetate (FDA), which is widely used for viability detection both of animal and plant cells, this compound passes through the cell membrane and is converted into a fluorescent substrate, named fluorescein, by the endogenous esterase enzyme. In general, the use of FDA makes it possible to distinguish living cells from dead cells by fluorescence detection equipment (Yamori et al. 2006).

For viability detection of whole plants, a colorimetric technology has been developed oriented to determine the stress-produced damage in fruit, vegetable, plant or flower, which is based on the difference in the production of volatile compounds like ethanol and aldehyde by plants upon suffering the damage. The method comprises covering the plant with an isolating cover and determining the change in the gases in the interior thereof, the gas detection method is of the colorimetric type, with reagents of the potassium dichromate type for determining volatile ethanol (U.S. Pat. No. 6,306,620).

Over the last years and thanks to the increase in the capacity to measure weak light signals (Ntziachristos et al. 2005, Fujimoto et al. 2000, Watanabe et al. 2007), there has been developed a technology for determination of seed viability by retarded luminescence. It is considered that photons may be considered as information carriers due to their interaction at the atomic and molecular level, providing information regarding the chemical components and the complex structure of the systems (Costanzo et al. 2008). Even though the system allows to determine the viability of plant samples non invasively and with quite accurate results, a series of sophisticated pieces of equipment are required for performing the measurements and the analysis thereof.

On the other side, for seed viability detection there exist some non destructive technologies based on bioelectric current. Each organism contains redox activity levels, and in plants, it has been established that bioelectric currents are associated with ion mobility. Redox activity can be used to monitor enzymatic activity levels and consequently seed viability. In the method designed, seeds are moistened to initiate the first phase of their pregermination cycle, and electric current is passed therethrough. Seed viability has been calibrated by correlating the radicular length with the electric current values measured in the corresponding seeds (U.S. Pat. No. 3,852,914). This method, although non invasive, requires special equipment to be able to perform the measurements and the analysis thereof.

This disclosure possesses advantages over the subject matters described in the state of the art, since it allows to detect in a simple manner the viability of plant cells, tissues and whole plants without damaging the analyzed samples, allowing the subsequent growth and utilization thereof in a normal manner. On the other side, this disclosure provides trustworthy results without requiring sophisticated equipment for the implementation thereof or for interpretation of results.

### SUMMARY

This disclosure relates to a viability detection method for plant cells, tissues and/or whole plants, based on the determination of the activity of the alpha-amylase released into the medium, the enzyme is indirectly detected by determining degraded starch in a culture medium supplemented with the carbohydrate.

The method for determining the viability of a plant sample comprises the following steps:
a) providing a viability detection device containing a solid or semisolid culture medium suitable for the nutritional requirements of a plant sample, wherein the culture medium has a starch supplement;
b) growing the plant tissue in the viability detection device from the previous step;
c) removing the plant tissue sample from the viability detection device;
d) revealing the viability detection device.

This disclosure comprises a viability detection kit for a plant sample, which comprises: a support, a culture medium, a starch supplement, and an iodine-based revealing composition, wherein the starch supplement is to be added to the culture medium in a concentration sufficient to achieve a starch concentration in the culture medium of between 0.5 and 5.0 gL⁻¹, and wherein the culture medium and the starch supplement are admixed to form a culture medium supplemented with starch. The support contains the culture medium supplemented with starch. The culture medium supplemented with starch has a starch concentration of between 0.5 and 5.0 gL⁻¹.

This disclosure comprises a viability detection device for a plant sample, which comprises a support which contains a culture medium supplemented with starch in which the plant tissue is grown.

The viability detection device comprises a support which comprises a plate, a flask, a Petri plate or an Eppendorf tube, with or without a lid.

The present viability detection method does not destroy or damage plant tissues, allowing the normal growth of the plant after being submitted to viability detection analysis.

### BRIEF DESCRIPTION OF THE DRAWINGS

FIG. 1 shows standardization of the starch concentration in the culture medium for detection of plant viability. The photograph shows 4 plates with culture medium (MS basal medium+7 gL⁻¹ of agar-agar+30 gL⁻¹ of sucrose) supplemented with different starch concentrations, Plate A: 1.0 gL⁻¹; B: 1.0 gL⁻¹, Plate C: 1.5 gL⁻¹, Plate D: 2.0 gL⁻¹. Explants of nodal segments of tobacco were cultured in the 4 plates for 72 hours at 25° C. in the dark. Subsequently, the explants were removed from plates B, C and D, and plates B, C and D were revealed with a 10% iodine solution. Plate A corresponds to controls of living explants which correspond to nodal segments of tobacco growing in MS+7 gL⁻¹ of agar-agar+30 gL⁻¹ of sucrose+starch at 1.0 gL⁻¹, which plate was not revealed. The arrows in plates B, C and D indicate some of the locations where the explants were seeded.

FIG. 2 shows viability detection in living (A) and dead (B) rhizomes of the terrestrial orchid Chloraea crispa. The sign of viability in the living tissues (Plate A) is expressed in the formation of a colorless halo in the zone where the explants were cultured, an effect that is not produced in dead tissues (Plate B). The living and dead tissues were incubated in plates A and B during 72 hours in the dark, at rest and at a temperature of 25° C. After that time, the rhizomes were removed and the plates were treated with revealing solution. The arrows indicate the locations where the explants were seeded.

FIG. 3 shows viability detection in different papaya tissues. Fig. A corresponds to detection of the viability of a nodal segment of papaya (Carica vasconcellea) cultured in MS basal medium+sucrose (30 gL⁻¹)+GA₃ (1.0 mgL⁻¹)+starch (1.0 gL⁻¹) in triplicate. Fig. B corresponds to detection of the viability of papaya (Carica vasconcellea) leaves cultured in MS basal medium+sucrose (30 gL⁻¹)+GA₃ (1.0 mgL⁻¹)+starch (1.0 gL⁻¹) in triplicate. All tissues were incubated in plates during 72 hours in the dark, at rest and at a temperature of 25° C. After that time, the nodal segments and the leaves from papaya were removed from the plates, and the plates were treated with revealing solution. The arrows indicate some of the locations where the explants were seeded.

FIG. 4 shows viability detection in orchid meristems. The tissues were cultured in Van Waes Medium salts+sucrose (30 gL⁻¹)+TDZ (1.5 mgL⁻¹)+IBA (1.5 mgL⁻¹)+starch (1.0 gL⁻¹) in triplicate. All tissues were incubated in the plates during 72 hours in the dark, at rest and at a temperature of 25° C. After that time, the meristems were removed, and the plates were treated with revealing solution. The arrow indicates one of the locations where the explants were seeded.

FIG. 5 shows viability detection in nodal segments of tobacco (Nicotiana tabacum). The tissues were cultured in MS+sucrose (30 gL⁻¹)+6-benzylaminopurine (BAP) (0.1 mgL⁻¹)+starch (1.0 gL⁻¹) in triplicate. All tissues were incubated in plates during 72 hours in the dark, at rest and at a temperature of 25° C. After that time, the nodal segments were removed and the plates were treated with revealing solution. The arrow indicates one of the locations where the explants were seeded.

FIG. 6 shows the effect of starch on the morphogenic response of nodal segments cultured in propagation basal medium for 15 days. (A) shows nodal segments of tobacco cultured in culture medium without a carbon source. (B) shows nodal segments of tobacco cultured in culture medium supplemented with sucrose at 30 gL⁻¹ as an energy source, and starch at 1.5 gL⁻¹. (C) shows nodal segments of tobacco cultured in culture medium supplemented only with starch at 1.5 gL⁻¹.

### DETAILED DESCRIPTION

The method proposed is based on detection of the activity of the alpha-amylase enzyme released into the culture medium, as a way of determining the viability of any plant tissue. In general terms, the method is based on the ability of the alpha-amylase enzyme for degrading the starch present in the culture medium of the detection device where the plant sample is grown, the degradation is evidenced by the lack of color on the surface of the culture medium of the device, upon being revealed with an iodine-based solution. The alpha-amylase enzyme is mainly present in plant tissues, whereby the method has a level of selection toward the growth of other organisms, without prejudice to the foregoing. Preferably, the method is developed under conventional aseptic conditions of the state of the art.

The alpha-amylase enzyme is present in all plant tissues and cells, and although it exhibits a different degree of presence and activity in the different tissues and stages of development of a plant, they are perfectly detectable through the proposed method.

The method disclosed allows to determine viability in plant samples from different species of gymnosperm or angiosperm, monocotyledonous or dicotyledonous plants, from plants grown in vivo or cultured in vitro. On the other side, the present method allows to determine viability at different stages of development of the plant, for example, and not limited to organogenesis, callogenesis, somatic embryogenesis, differentiated tissues and sex tissues. The method for determining viability allows to determine viability in plant tissues from different parts of the plant, not limited to leaves, stems, petioles, calluses, embryos, protocorms, rhizomes or roots, in addition to polen and seeds.

The method for determining viability as designed may be applied to any plant culture process which requires a viability detection step either with commercial, scientific research or other purposes, for example, and not limited to, micropropagation technology, viability in polen grains, viability of in vitro and ex vitro tissues submitted to biotic and abiotic stress, viability of tissues from genetically engineered plants, viability of ovaries, detection of the viability of any plant tissue and culturing in bioreactors.

The culture medium is chosen from the state of the art according to the nutritional requirements typical of the plant tissue to be analyzed. The culture medium may be supplemented with compounds normally used in processes such as: culturing plant tissues; selecting transformed tissues; avoiding contamination of the medium with other prokaryotic or eukaryotic organisms; stimulating the morphogenic response of the tissues; ensuring the development and growth of the cells or tissues in the physiologic state in which the present viability detection method is developed, among others. The compounds with which the culture medium may be supplemented comprise, for example: inorganic salts, organic salts, minerals, vitamins, aminoacids, natural or synthetic growth regulators, agar or any other polymer used to solidify culture media, bactericides, fungicides; organic acids and inorganic acids and water.

The culture medium is additionally supplemented with starch. Preferably, the starch concentration of the culture medium is between 0.5 and 5.0 gL⁻¹, preferably between 1.0 and 3.0 gL⁻¹, more preferably between 1.0 and 2.0 gL⁻¹.

The revealing composition is based on iodine with a 10% iodine solution, the revealing composition may further contain preservatives, such as organic acids, antibiotics and fungicides.

The device is manufactured under regular sterility and asepsis conditions described in the state of the art. The plant sample is treated considering the normal aseptic conditions described in the state of the art.

In the method for determining viability, the plant samples are placed on the surface of the culture medium of the device and are incubated for a period of time and in temperature conditions suitable for each plant species. Preferably, the culturing of plant tissues is carried out in dark conditions since a greater starch uptake by the plant tissue is obtained.

In step c) the tissue sample is removed from the culture medium. The analyzed plant sample may be subsequently subcultured in culture medium without starch or used according to the purposes deemed convenient by the user.

In step d) for revealing the viability detection device, an iodine solution is poured onto the surface of the culture medium which is incubated for a period of time at room temperature. Preferably, this incubation lasts between 3 and 5 minutes and is conducted between 20 and 25° C. Subsequently, the iodine solution is removed from the surface and, as a positive viability result, a colorless halo is observed in the location where the plant sample was grown. This halo reflects the degradation of the starch present in the culture medium. Degradation of the starch by the enzymes of the plant is evidenced visually, thus detecting the viability of the assessed sample. If the tissue is alive, a colorless halo is observed underneath the place where the tissue was located. The halo may be of a variable size, depending on the type of tissue and the plant species, however, clear differences are observed between the color of the culture medium and the halo formed in the zone where the explant was cultured, when the tissue is viable. If the tissue is dead, the surface underneath the evaluated explant is stained with blue similar to the rest of the culture medium.

### EXAMPLES

### Example 1

### Standardization of the Culture Medium for the Viability Detection Device

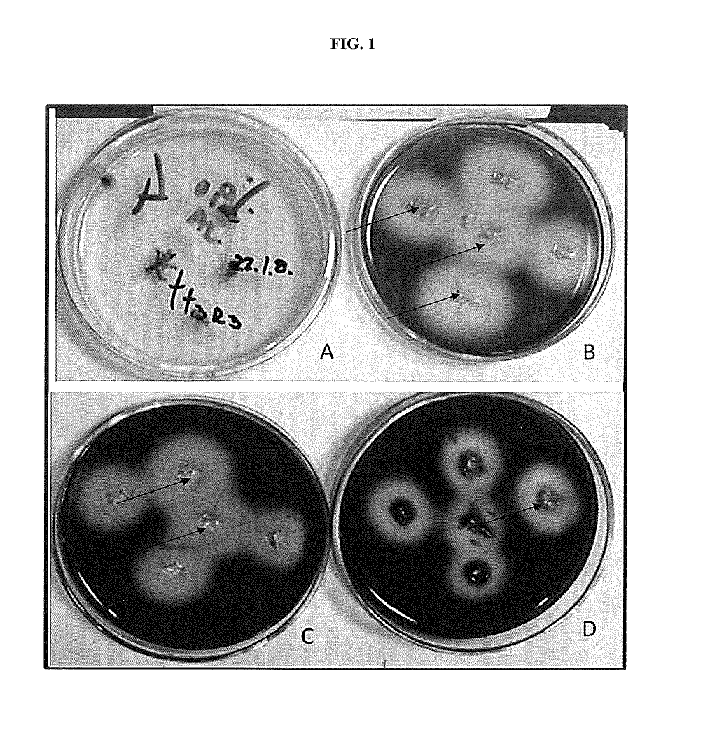

To define the starch composition of the test culture medium to be used in the following experiments, tests were conducted with basic culture medium supplemented with different starch concentrations. In this experiment (FIG. 1), nodal segments of tobacco (Nicotiana tabacum) were used as a model.

The culture medium was prepared with MS basal medium (Murashige and Skoog, 1962) and agar-agar (7 gL⁻¹), supplemented with sucrose (30 gL⁻¹) and different amounts of starch (0.5 gL⁻¹, 1.0 gL⁻¹, 1.5 gL⁻¹, 2.0 gL⁻¹). The pH of the culture medium was adjusted to 5.6-5.7, before sterilizing. Sterilization of the culture medium was performed by pressurized steam in an autoclave at a temperature of 121° C., a pressure of 1 kgcm⁻² and for 30 minutes. Once sterile, the culture media were dispensed into viability detection devices such as Petri plates in aseptic conditions.

The explants were taken from aseptic nodal segments of tobacco grown in vitro. The explants were prepared in segments of 0.5 cm in length and 0.5 cm in length. Once prepared, the explants were placed in the viability detection devices, trying to keep sufficient distance therebetween to avoid interferences in the signs of viability, preferably 5 explants per device. The explants were cultured for 1, 2, 3 and 5 days, at 25° C. and in dark conditions. After this incubation period, the explants were removed from the plates and the latter were revealed with an iodine solution as indicated in the following paragraph.

Revealing of the plates was performed using an iodine solution diluted to 10%. The iodine solution diluted to 10% was prepared in two steps: firstly, a colorless solution of potassium iodide (KI) at 300 gL⁻¹ was prepared. Subsequently, the KI solution was used to prepare the iodinated solution by adding 233.1 mL of KI solution and 56 g of iodine crystals to 500 ml of distilled water. The solution was stirred for 1 hour or until the crystals were completely dissolved and the solution was homogenized, and was left to stand for 24 hours. Finally, the volume of the solution was adjusted by adding 3.5 liters of distilled water.

To visualize the signs of viability in the detection plates, a film of iodine solution diluted to 10% was applied for 3 minutes on the surface of the plates until staining was observed in the culture medium. Subsequently, the iodine solution was removed from the surface of the plates by runoff.

FIG. 1 shows the results of the standardization of the culture medium in viability detection experiments on nodal segments of tobacco, in plates B, C and D, a colorless halo is observed on the surface where the implants were cultured, demonstrating the viability of the grown tissues. It was determined to use 1.5 gL⁻¹ of starch in the culture medium as the preferred concentration for cell viability detection in this experimental model.

### Example 2

### Viability Detection in Different Explants from Strawberry (Fragaria chiloensis), Tobacco (Nicotiana tabacum), Blueberries (Vaccinum corymbosun) and Andean Papaya (Carica vasconcellea)

Viability tests were conducted on explants obtained from different plant species and different tissues thereof, using our viability detection device.

The experimental steps for detecting the viability of the different tissues are described below:

### a) Preparation of the Viability Detection Plates:

MS basal medium (Murashige and Skoog, 1962) was used, supplemented with 1.5 gL⁻¹ of starch, 30 gL⁻¹ of sucrose, 7 gL⁻¹ of agar-agar and growth regulators according to the type of explant and the species, as indicated in Table 1. This working concentration was chosen according to the type of explant, the plant species and the morphogenic process in which the viability detection is performed.
The basal salts and the vitamins of the MS medium were added according to the concentrations suggested for the preparation of this culture medium, without any modification (Murashige and Skoog, 1962). The pH of the culture medium was adjusted to 5.6-5.7, before sterilizing. Sterilization of the culture medium was performed by pressurized steam in an autoclave at a temperature of 121° C., a pressure of 1 kgcm⁻² and for 30 minutes. Once sterile, the culture media were dispensed into Petri plates in aseptic conditions.
The plates may be sealed and kept in the dark and at room temperature for up to 30 days before being used.

TABLE 1
Composition of the culture media used in the viability tests on different explants and species.

| Species | Explant | Culture medium |
|---|---|---|
| Tobacco | Leaves, stems, | MS salts + sucrose (30 gL⁻¹) + 6-benzyl-aminopurine (BAP) (0.1 mgL⁻¹) |
| | petioles, calluses | |
| Papaya | Leaves, stems, | MS salts + sucrose (30 gL⁻¹) + gibberellic acid (GA₃) (1.0 mgL⁻¹) |
| | petioles | |
| | Calluses | MS salts + sucrose (30 gL⁻¹) + 2,4-dichloro-phenoxyacetic acid (2,4-D) |
| | | (1.0 mgL⁻¹) + thidiazuron (TDZ) (1.5 mgL⁻¹) |
| Strawberry | Leaves, stems, | MS salts + sucrose (30 gL⁻¹) + indole-butyric acid (IBA) (1.0 mgL⁻¹) |
| | petioles | |
| | Calluses | MS salts + sucrose (30 gL⁻¹) + IBA (0.01 mgL⁻¹) + TDZ (0.25 mgL⁻¹) |
| Blueberries | Leaves, stems, | Woody Plant Medium salts (McCown and Lloyd, 1991) + sucrose |
| | petioles, calluses | (30 gL⁻¹) + 2-iP (4.0 mgL⁻¹). |
| Orchids | Leaves, rhizomes | Van Waes Medium salts (van Waes and Debergh, 1986) + sucrose |
| | | (30 gL⁻¹) + TDZ (1.5 mgL⁻¹) + IBA (1.5 mgL⁻¹). |
| | Calluses, somatic | Van Waes Medium salts (van Waes and Debergh, 1986) + sucrose |
| | embryos | (30 gL¹) + BAP (0.2 mgL⁻¹). |

### b) Preparation of the Explants:

All explants were taken from plants grown under in vitro conditions. The explants were prepared in segments of 0.5 cm in length and 0.5 cm in length in the case of the leaves; 0.5 cm in diameter for the calluses; 0.5 cm in length for the nodal segments and petioles.

### c) Culturing of the Explants in the Culture Plates and Viability Assay.

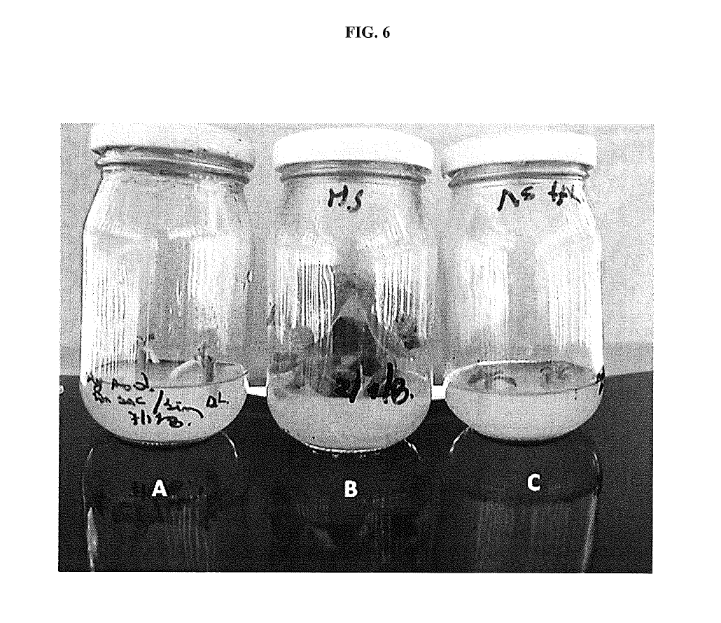

Once prepared, the explants were placed in the viability detection plate, trying to keep sufficient distance therebetween to avoid interferences in the signs of viability. In this case, not more than 6 explants were placed in plates of 10 cm in diameter, without prejudice to other densities in the species and tissues that might permit them. All the explants were placed on the culture medium as if they were to be manipulated for generating morphogenic responses according to the indications of the protocols for each species.
The explants were cultured in the viability detection plates from 24 hours to 5 days and were cultured at 25° C. and in dark conditions.
The explants were removed from the viability detection medium for the subsequent revealing of the plates. The explants were subcultured in a culture medium recommended for each species to induce the desired morphogenic response. In this case, the basal culture medium described in Table 1 was used, but without the addition of starch. FIG. 6 shows that the culturing period in the viability detection medium did not affect the morphogenic response.

Revealing of the viability detection plates was performed using an iodine solution diluted to 10%. This solution was prepared according to the protocol for the preparation of the iodine solution described in Example 1.

To visualize the signs of viability in the detection plates, a film of iodine solution diluted to 10% was applied for 3 minutes on the surface of the plates until staining was observed in the culture medium. Subsequently, the iodine solution was removed from the surface of the plates by runoff.

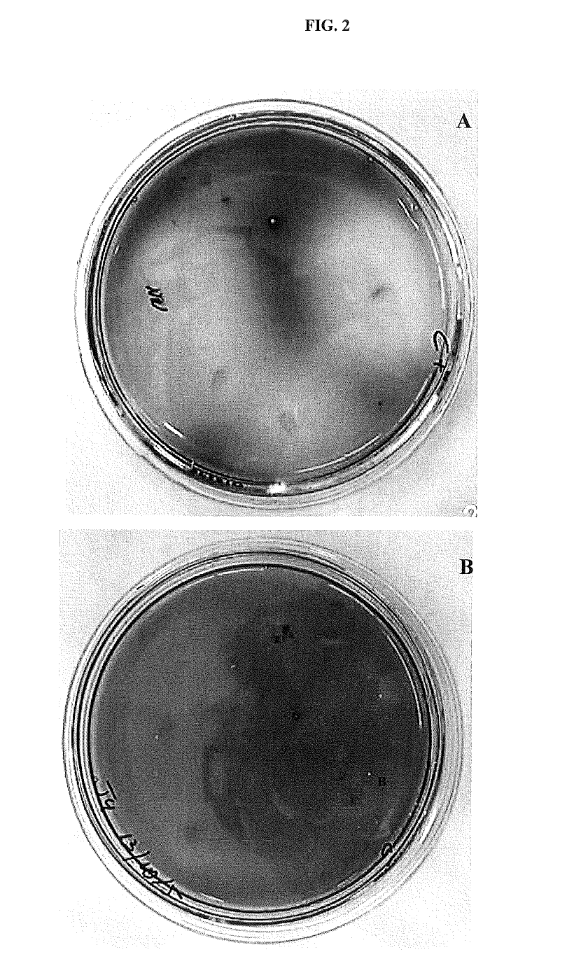

As a way of control, viability tests were carried out with samples of living and dead tissues. To this end, rhizomes of the terrestrial orchid Chloraea crispa cultured in vitro were used. The dead tissue samples were obtained by sterilizing them in an autoclave at 121° C. and 1 kgcm⁻² for 40 minutes. FIG. 2 shows the results of the experiment, demonstrating that the detection system allows to clearly determine the viability of the tissue under study, since a colorless halo can be clearly observed on the surface of the plate where the living tissues were cultured (FIG. 2A), due to the degradation of the starch in this sector of the plate. The halos are not observed in the plates with dead tissues (FIG. 2B).

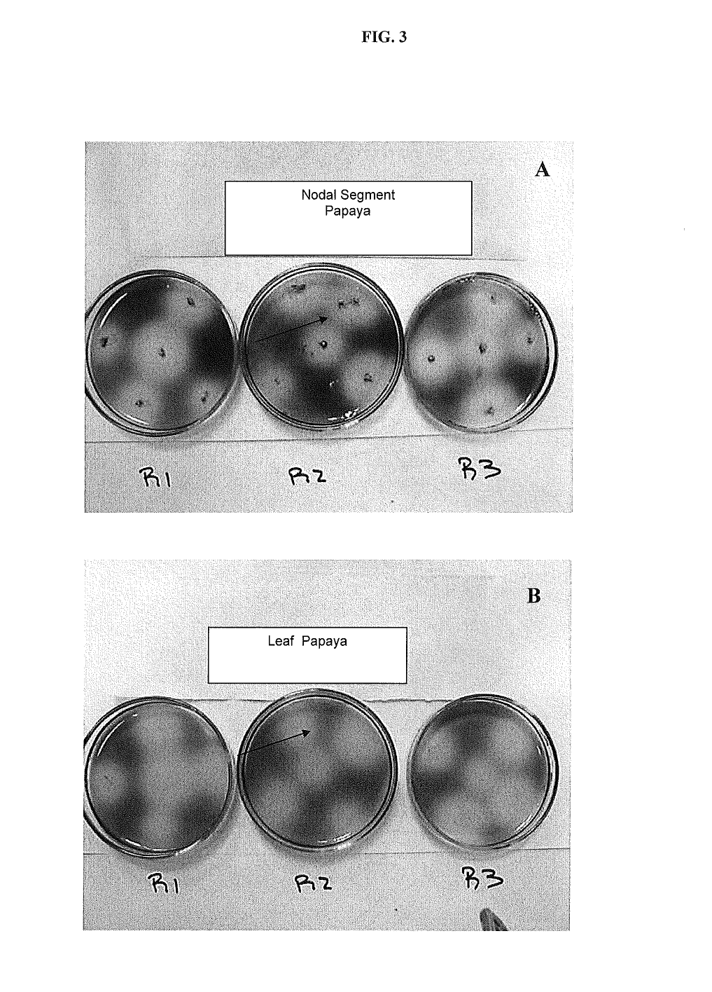

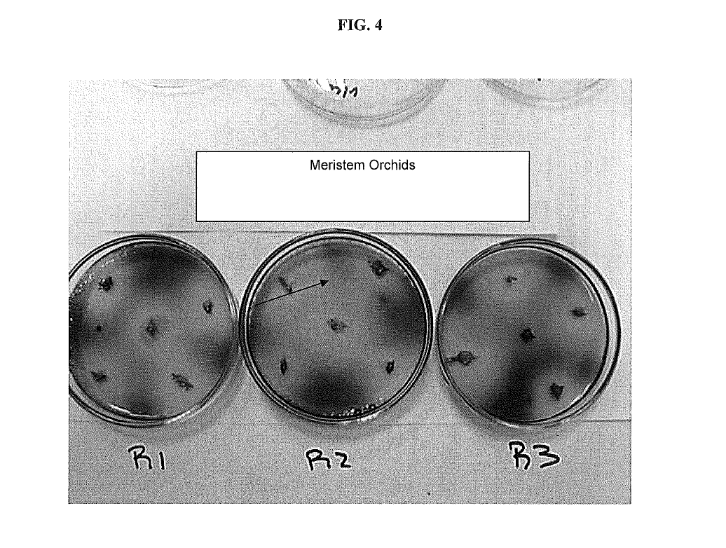

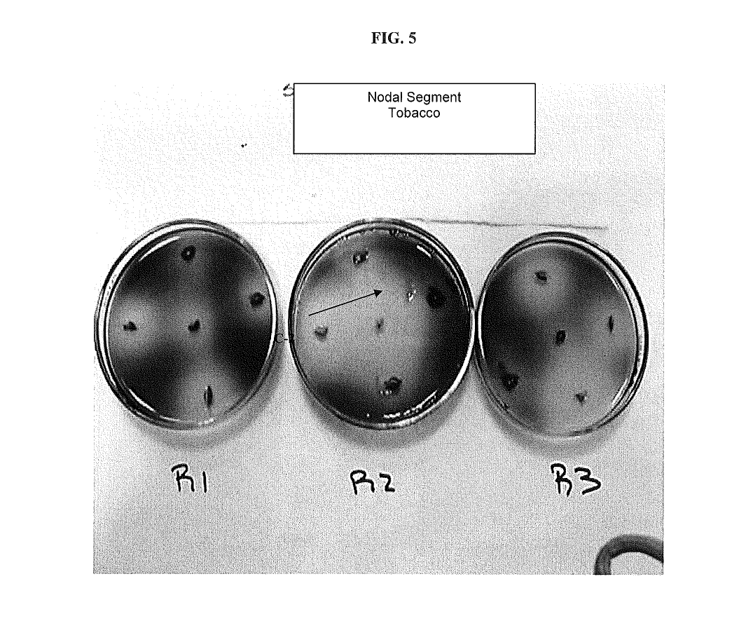

FIGS. 3, 4 and 5 show the results of the viability tests performed for some of the types of tissues described in Table 1. In these photographs, it can be confirmed that the system proposed allows to detect the activity of living tissues of different origins, both at the level of species and of tissue type, independent of the basal culture medium used.

It should be mentioned that the tissues used in these tests are those commonly used in the in vitro multiplication protocols of most plant species.

### Example 3

### Effect of Starch on the Growth of Plant Tissues

To determine the possible effect of starch on the normal growth and morphogenic response of the tissues submitted to the viability detection test, nodal segments of tobacco were cultured for 15 days in basal propagation medium (MS medium) supplemented with different carbon sources. The culture media were prepared according to the protocol described in the preceding examples. Three different culture media were prepared, which were: without carbon source (FIG. 6A), culture medium supplemented with sucrose at 30 gL⁻¹ as an energy source, and starch at 1.5 gL⁻¹ (FIG. 6B) and culture medium supplemented only with starch at 1.5 gL⁻¹ (FIG. 6C).

FIG. 6 shows the effect of the different types of culture media on nodal segments of tobacco. In the photographs of FIGS. 6A and 6C, scarce growth of the plants is observed, showing that starch does not replace the carbon source for these cultures. On the other side, in FIG. 6B it can be appreciated that starch did not affect the growth of the cultured plant, and the growth and morphogenic response produced were normal for the plant.

1. A method for determining the viability of a plant sample, comprising:
a) providing a viability detection device containing a solid or semisolid culture medium suitable for the nutritional requirements of a plant sample, wherein the culture medium has a starch supplement;
b) growing the plant tissue in the viability detection device from the previous step;
c) removing the plant tissue sample from the viability detection device; and
d) revealing the viability detection device.

2. The method of claim 1, wherein step a) comprises providing a viability device comprising a culture medium supplemented with starch at a starch concentration of between 0.5 and 5.0 gL-1, preferably between 1.0 and 3.0 gL-1.

3. The method of claim 1, wherein step a) comprises providing a viability device comprising a culture medium supplemented with starch at a starch concentration of between 1.0 and 2.0 gL-1, preferably 1.5 gL-1.

4. The method according to claim 1, wherein step b) is performed in dark conditions, at 25° C., during 72 hours.

5. The method according to claim 1, wherein the sample that is removed in step c) may be subsequently subcultured in a culture medium without starch.

6. The method according to claim 1, wherein the revealing step is performed with a revealing solution which is a 10% iodine solution, and which may optionally contain preservatives such as organic acids, antibiotics and fungicides.

7. The method according to claim 1, wherein in the revealing step a 10% iodine solution is poured onto the detection device and it is incubated for 3 to 5 minutes at a temperature of between 20 and 25° C.

8. The method according to claim 1, wherein the revealing step further comprises removing the iodine solution and observing the presence of a colorless halo which indicates that the plant sample is viable.

9. A viability detection device, comprising a support that possesses a culture medium supplemented with starch.

10. The viability detection device according to claim 9, wherein the culture medium has a starch concentration of between 0.5 and 5.0 gL-1, preferably between 1.0 and 3.0 gL-1.

11. The viability detection device according to claim 9, wherein the medium has a starch concentration of between 1.0 and 2.0 gL-1, preferably 1.5 gL-1.

12. The viability detection device according to claim 9, comprising a plate, a flask, a Petri plate or an Eppendorf tube, with or without a lid.

13. A viability detection kit, comprising a support, a culture medium, a starch supplement and a revealing composition.

14. The kit according to claim 13, wherein the starch supplement is to be added to the culture medium at a concentration sufficient to achieve a starch concentration in the culture medium of between 0.5 and 5.0 gL-1.

15. The kit according to claim 13, wherein the culture medium and the starch supplement are admixed to form a culture medium supplemented with starch.

16. The kit according to claim 13, wherein the support contains a culture medium supplemented with starch.

17. The kit according to claim 13, wherein the culture medium supplemented with starch has a starch concentration of between 1.0 and 3.0 gL-1.

18. The kit according to claim 13, wherein the starch concentration in the culture medium is between 1.0 and 2.0 gL-1, preferably 1.5 gL-1.

19. The kit according to claim 13, wherein the revealing solution is a 10% iodine solution, and may optionally contain preservatives such as organic acids, antibiotics and fungicides.

20. The kit according to claim 13, comprising a plate, a flask, a Petri plate or an Eppendorf tube, with or without a lid.
